(19) United States
(12) Patent Application Publication
(10) Pub. No.: US 2026/0194480 A1
(43) Pub. Date: Jul. 9, 2026

(54) DISPLACEMENT TEST DEVICE AND DISPLACEMENT TEST METHOD

(71) Applicant: China University of Geosciences (Wuhan), Wuhan City (CN)

(72) Inventors: Tao LONG, Wuhan City (CN); Yankun SUN, Wuhan City (CN); Peng PENG, Wuhan City (CN); Tianyu SUN, Wuhan City (CN); Liangchang ZHOU, Wuhan City (CN)

(21) Appl. No.: 19/082,395

(22) Filed: Mar. 18, 2025

(30) Foreign Application Priority Data
Jan. 9, 2025 (CN) 202510031417.2

(51) Int. Cl. G01N 24/08; G01N 15/08; G01N 33/24

(52) U.S. Cl. CPC G01N 24/081; G01N 15/082; G01N 15/088; G01N 33/241

(57) ABSTRACT

A displacement test device and a displacement test method are provided, relating to the technical field of displacement tests. The displacement test device includes a displacement fluid system, a core holder, and a nuclear magnetic resonance instrument. A fluid outlet of the displacement fluid system communicates with a fluid inlet of the core holder, and a core is arranged in the core holder. The displacement fluid system can transport a displacement fluid into the core holder, the core holder can displace a target fluid from the core, and the core holder is arranged in a detection cavity of the nuclear magnetic resonance instrument. According to the displacement test device and the displacement test method, shale can be detected during shale displacement test, and a displacement test effect can be evaluated well.

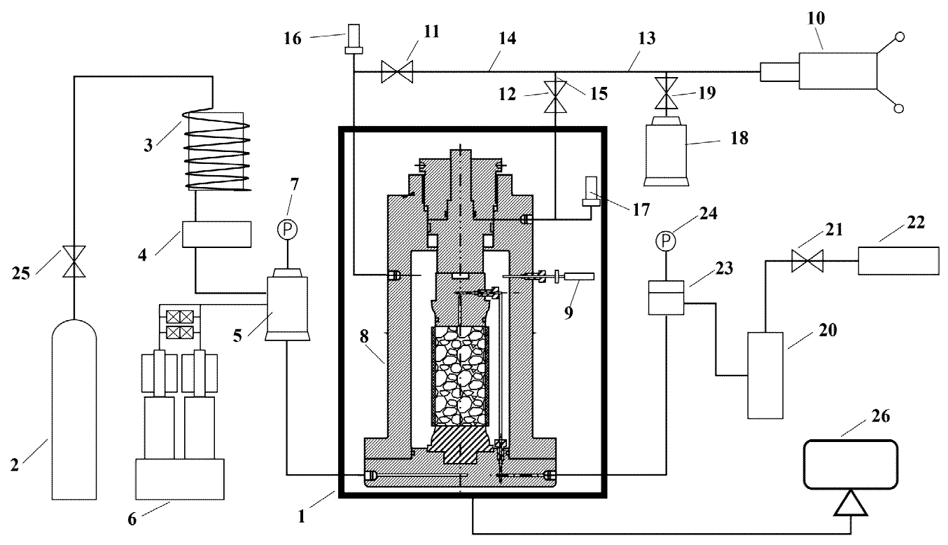

### CROSS-REFERENCE TO RELATED APPLICATION

This patent application claims the benefit and priority of Chinese Patent Application No. 202510031417.2 filed with the China National Intellectual Property Administration on Jan. 9, 2025, the disclosure of which is incorporated by reference herein in its entirety as part of the present application.

### TECHNICAL FIELD

The present disclosure relates to the technical field of displacement tests, and in particular to a displacement test device and a displacement test method.

### BACKGROUND

A displacement device is a test device that uses a displacement fluid to displace a target fluid based on infiltration effect at a certain temperature and pressure. However, most existing displacement devices only detect the displacement results, there is relatively little research on the changes of various parameters and indexes during shale displacement, and the effect of the displacement test cannot be evaluated well. Therefore, there is an urgent need of a displacement test device and displacement test method for detecting shale during shale displacement test, and evaluating a displacement test effect well.

### SUMMARY

An objective of the present disclosure is to provide a displacement test device and a displacement test method to solve the problems in the prior art. The shale can be detected during shale displacement test, and a displacement test effect can be evaluated well.

To achieve the objective above, the present disclosure employs the following technical solution.

The present disclosure provides a displacement test device, including a displacement fluid system, a core holder, and a nuclear magnetic resonance instrument. A fluid outlet of the displacement fluid system communicates with a fluid inlet of the core holder, and a core is arranged in the core holder. The core holder can displace a target fluid from the core by using a displacement fluid, and the core holder is arranged in a detection cavity of the nuclear magnetic resonance instrument.

Preferably, the displacement fluid system includes a power pump, and a material storehouse, a heating device, a purifier, a first container and the power pump which are connected and in communication in sequence. An output end of the power pump communicates with the first container, and a first pressure gauge is arranged on the first container.

Preferably, the core holder includes a holder body, a temperature detection device, and a pressure control assembly. The pressure control assembly is connected to the holder body, the core is arranged in the holder body, and the holder body can apply an axial pressure and a confining pressure to the core in the holder body. The pressure control assembly can control the magnitude of the confining pressure and the axial pressure of the holder body exerted on the core, a detection end of the temperature detection device is located in the holder body, and the temperature detection device can detect a temperature in the holder body.

Preferably, the pressure control assembly includes a pressure control pump, a first valve, a second valve, a main pipeline, a first branch pipe, and a second branch pipe. One end of the main pipeline communicates with an output end of the pressure control pump, one end of the first branch pipe communicates with the main pipeline, and another end of the first branch pipe communicates with a confining pressure regulation port of the holder body. One end of the second branch pipe communicates with one end, away from the pressure control pump, of the main pipeline, and another end of the second branch pipe communicates with an axial pressure regulation port of the holder body. The first valve is arranged on the first branch pipe, and the second valve is arranged on the second branch pipe.

Preferably, the pressure control pump is a manual pump.

Preferably, the pressure control pump further includes a first pressure relief pipeline, and a second pressure relief pipeline. One end of the first pressure relief pipeline communicates with the first branch pipe between the first valve and the holder body, and another end of the first pressure relief pipeline is provided with a first pressure relief valve. One end of the second pressure relief pipeline communicates with the second branch pipe between the second valve and the holder body, and another end of the second pressure relief pipeline is provided with a second pressure relief valve.

Preferably, the pressure control assembly further includes a second container, a third branch pipe, and a third valve. One end of the third branch pipe communicates with the main pipeline, another end of the third branch pipe communicates with the second container, and the third valve is arranged on the third branch pipe.

Preferably, the displacement test device further includes a fluid treatment system. The fluid treatment system includes a gas-liquid separator, a fourth valve, and a gas flowmeter. A fluid output end of the core holder communicates with the gas-liquid separator, a gas output end of the gas-liquid separator communicates with the fourth valve, and the fourth valve communicates with the gas flowmeter.

Preferably, the fluid treatment system further includes a back pressure valve, a fluid output end of the core holder communicates with the back pressure valve, and the back pressure valve communicates with a fluid inlet of the gas-liquid separator.

The present disclosure further provides a displacement test method, including the following steps:
initial detection: placing a treated core in a core holder, and detecting pore size distribution of a core without displacement test by a nuclear magnetic resonance instrument; and
test stage: displacing a fluid into the core holder through a displacement fluid system, displacing a target fluid from the core by using the displacement fluid, and detecting the pore size distribution of the core by using the nuclear magnetic resonance instrument.

Compared with the prior art, the present disclosure has the following technical effects:

According to the displacement test device provided by the present disclosure, during test, a gas in the displacement fluid system enters the core holder, the core holder can displace a target fluid from the core by using the displacement fluid, thus completing the displacement work. During displacement, the nuclear magnetic resonance instrument can continuously detect the core, thus obtaining pore size distribution data of the core during displacement test and evaluating the effect of the displacement test better.

Further, in the present disclosure, the control of the confining pressure and the axial pressure is more convenient by arranging the first pressure relief valve and the second pressure relief valve on the first pressure relief pipeline and the second pressure relief pipeline, respectively.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

The FIGURE is a schematic structural diagram of a displacement test device according to the present disclosure.

In the drawings: 1—nuclear magnetic resonance instrument; 2—material warehouse; 3—heating device; 4—purifier; 5—first container; 6—power pump; 7—first pressure gauge; 8—holder body; 9—temperature detection device; 10—pressure control pump; 11—first valve; 12—second valve; 13—main pipeline; 14—first branch pipe; 15—second branch pipe; 16—first pressure relief valve; 17—second pressure relief valve; 18—second container; 19—third calve; 20—gas-liquid separator; 21—fourth valve; 22—gas flowmeter; 23—back pressure valve; 24—second pressure gauge; 25—fifth valve; 26—computer.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

An objective of the present disclosure is to provide a displacement test device and a displacement test method to solve the problems in the prior art. The shale can be detected during shale displacement test, and a displacement test effect can be evaluated well.

In order to make the objectives, features and advantages of the present disclosure more clearly, the present disclosure is further described in detail below with reference to the embodiments.

### Embodiment 1

This embodiment provides a displacement test device, as shown in the FIGURE, including a displacement fluid system, a core holder, and a nuclear magnetic resonance instrument. A fluid outlet of the displacement fluid system communicates with a fluid inlet of the core holder, and a core is arranged in the core holder. The displacement fluid system can transport a displacement fluid into the core holder, the core holder can displace a target fluid from the core, and the core holder is arranged in a detection cavity of the nuclear magnetic resonance instrument.

According to the displacement test device provided in this embodiment, during test, the fluid in the displacement fluid system enters the core holder, the core holder is used to apply an axial pressure and a confining pressure to the core, and can displace a target fluid from the core by using a displacement fluid, thus completing the displacement work. During displacement, the nuclear magnetic resonance instrument 1 can continuously detect the core, thus obtaining pore size distribution data of the core in the displacement test process, and evaluating the effect of the displacement test better. The nuclear magnetic resonance instrument 1 is in signal connection with a computer 26, and the nuclear magnetic resonance instrument 1 can perform T2 spectrum test on the core and output the data to the computer 26. The displacement fluid is preferably CO₂, or may also be light hydrocarbon or water.

In a preferred embodiment of this embodiment, the displacement fluid system includes a power pump 6, and a material storehouse 2, a heating device 3, a purifier 4 and a first container 5 which are connected and in communication in sequence. An output end of the power pump 6 communicates with the first container 5, and a first pressure gauge 7 is arranged on the first container 5. A fluid inlet of the core holder has two states of opening, and closing. When the displacement fluid is introduced into the core holder, the fluid inlet of the core holder is closed first, and the displacement fluid is introduced into the heating device 3 which is used to heat the displacement fluid to a temperature required by the test, and then the heated displacement fluid is introduced into the purifier which is used to purify and remove impurities from the displacement fluid to ensure the purity of the displacement fluid. Afterwards, the gas after impurity removal is introduced into the first container 5, and the first pressure gauge 7 on the first container 5 is observed. If there is a digital display on the first pressure gauge 7, it is indicated that there is no damage and leakage on all parts. In this case, the fluid inlet of the core holder is opened, and the fluid is introduced into the core holder by the power pump 6 for the displacement test. The power pump 6 is preferably a constant-pressure constant-speed pump, and a fifth valve 25 is arranged between the material warehouse 2 and the heating device 3.

In a preferred embodiment of this embodiment, the core holder includes a holder body 8, a temperature detection device 9, and a pressure control assembly. The pressure control assembly is connected to the holder body 8, the core is arranged in the holder body 8, and the holder body 8 can apply an axial pressure and a confining pressure to the core in the holder body 8. The pressure control assembly can control the magnitude of the confining pressure and the axial pressure of the holder body 8 on the core. A detection end of the temperature detection device 9 is located in the holder body 8, and the temperature detection device 9 can detect a temperature in the holder body 8. After a fluid inlet of the displacement system is opened, whether the temperature on the temperature detection device 9 reaches the temperature of the heating device 3 for heating the displacement fluid is observed. If the temperature on the temperature detection device 9 reaches the temperature of the heating device 3 for heating the displacement fluid, it is indicated that the fluid volume of the displacement fluid in the holder body 8 has reached the test requirements (if not, the displacement fluid is continuously introduced), then the core holder is opened and the pressure control assembly is adjusted to apply the axial pressure and confining pressure required by the test to the core, thus carrying out the displacement work.

It should be noted that the holder body in the present disclosure is the prior art known to the inventor, and the application of the axial pressure and the confining pressure is achieved by the existing fluid pressurization method.

In a preferred embodiment of this embodiment, the pressure control assembly includes a pressure control pump 10, a first valve 11, a second valve 12, a main pipeline 13, a first branch pipe 14, and a second branch pipe 15. One end of the main pipeline 13 communicates with an output end of the pressure control pump 10, one end of the first branch pipe 14 communicates with the main pipeline 13, and the other end of the first branch pipe 14 communicates with a confining pressure regulation port of the holder body 8. One end of the second branch pipe 15 communicates with one end, away from the pressure control pump 10, of the main pipeline 13, and the other end of the second branch pipe 15 communicates with an axial pressure regulation port of the holder body 8. The first valve 11 is arranged on the first branch pipe 14, and the second valve 12 is arranged on the second branch pipe 15. During pressure adjustment, if the confining pressure needs to be adjusted, the first valve 11 is opened, the second valve 12 is closed, and the fluid is introduced into the main pipeline and the first branch pipe through the pressure control pump 10 until a preset confining value is reached. During axial pressure adjustment, the second valve 12 is opened, the first valve 11 is closed, the fluid is injected into the main pipeline and the second branch pipe 15 by the pressure control pump 10 until a preset axial pressure value is reached. The control of the axial pressure and the confining pressure is more convenient through the pressure control pump 10, the first valve 11 and the second valve 12. The pressure control pump 10 can inject gas or water into the confining pressure regulation port and the axial pressure regulation port of the holder body 8 as long as the filling pressure and the requirements of the displacement test can be satisfied.

In a preferred embodiment of this embodiment, the pressure control pump 10 is a manual pump. The pressure control pump 10 is preferably a manual air pump. Compared with a hydraulic oil pump and other devices, the manual pump is more convenient to operate and can reduce the mechanical failure rate.

In a preferred embodiment of this embodiment, the pressure control assembly further includes a first pressure relief pipeline, and a second pressure relief pipeline. One end of the first pressure relief pipeline communicates with the first branch pipe 14 between the first valve 11 and the holder body 8, and the other end of the first pressure relief pipeline is provided with a first pressure relief valve 16. One end of the second pressure relief pipeline communicates with the second branch pipe 15 between the second valve 12 and the holder body 8, and the other end of the second pressure relief pipeline is provided with a second pressure relief valve 17. During the test, the axial pressure and the confining pressure sometimes need to be adjusted during displacement. For example, the initial axial pressure and confining pressure are both 8 MPa. After the test is carried out for 5 minutes, the axial pressure and the confining pressure need to be reduced to 5 MPa. At this time, the first pressure relief valve 16 and the second pressure relief valve 17 can be opened, and then the relief valves can be closed after the axial pressure and the confining pressure are reduced to 5 MPa. The control of the confining pressure and the axial pressure is more convenient by arranging the first pressure relief valve 16 and the second pressure relief valve 17. A pressure sensor is arranged in the holder body 8, which can be used to detect the magnitude of the axial pressure and confining pressure.

In a preferred embodiment of this embodiment, the pressure control assembly further includes a second container 18, a third branch pipe, and a third valve 19. One end of the third branch pipe communicates with the main pipeline 13, the other end of the third branch pipe communicates with the second container 18, and the third valve 19 is arranged on the third branch pipe. Before adjusting the axial pressure and the confining pressure, the first valve 11 and the second valve 12 are closed, and then the third valve 19 is opened. The fluid is introduced into the second container 18 by the pressure control pump 10. After the second container 18 is full of fluid, the third valve 19 is closed, and the axial pressure and the confining pressure are regulated by using the first valve 11, the second valve 12 and the pressure control pump 10. By providing the second container 18, the unstable pressure when the pressure-controlled pump 10 is initially ventilated is buffered, thus preventing the test from pressure fluctuation, and improving the accuracy of the test.

In a preferred embodiment of this embodiment, the displacement test device further includes a fluid treatment system. The displacement fluid is carbon dioxide. The fluid treatment system includes a gas-liquid separator 20, a fourth valve 21, and a gas flowmeter 22. A fluid output end of the core holder communicates with the gas-liquid separator 20, a gas output end of the gas-liquid separator 20 communicates with the fourth valve 21, and the fourth valve 21 communicates with the gas flowmeter 22. After the displacement system completes the displacement work, the displacement fluid is introduced into the gas-liquid separator 20, the gas-liquid separator 20 is used to separate the gas and liquid in the replacement fluid, the liquid remains in the gas-liquid separator 20, and the gas enters the gas flowmeter 22. When a numerical value in the gas flowmeter 22 no longer changes, it is indicated that the displacement work is completed (if the specific time of the displacement work is specified in the test, the specific time of the displacement work shall prevail). When there is no fluid treatment system, other containers can be used to collect the displacement fluid, and gas-liquid separation and metering can be carried out in other devices.

In a preferred embodiment of this embodiment, the fluid treatment system further includes a back pressure valve 23. A fluid output end of the core holder communicates with the back pressure valve 23, and the back pressure valve 23 communicates with a fluid inlet of the gas-liquid separator 20. When the fluid pressure at the back pressure valve 23 is less, the gas can pass through the back pressure valve 23 and flow into the gas-liquid separator. When the fluid pressure is large, the back pressure valve 23 is opened, the fluid is discharged to reduce the pressure, and the security of the device is improved. A second pressure gauge 24 is arranged on the back pressure valve 23.

### Embodiment 2

The present disclosure further provides a displacement test method, including the following steps:

Initial detection: A treated core is placed in a core holder, and a nuclear magnetic resonance instrument is used to detect pore size distribution of a core without displacement test.

Test stage: a fluid is displaced into the core holder through a displacement fluid system, the displacement fluid is used to displace a target fluid from the core, and the nuclear magnetic resonance instrument is used to detect pore size distribution of the core.

According to the displacement test method provided by this embodiment, the nuclear magnetic resonance spectrometer 1 can be used to detect pore size distribution of the core during the displacement test, thus evaluating a displacement test effect better.

Specifically, the test can be carried out according to the following steps:

Preparation step: A core is dried in an oven at 120° C. for 72 hours to evaporate moisture in a core, the core is saturated with oil for 48 hours after vacuumizing the core by a vacuum pressure saturator, and the core is measured by a nuclear magnetic resonance instrument 1 to obtain an initial T2 spectrum curve.

Pressure regulation: The core is placed in a holder body 8, a first valve 11 and a second valve 12 are closed and a third valve 19 is closed; the second container 18 is pressurized by a pressure control pump 10 until the pressure is full (a handle of the manual pump cannot rotate continuously); then the third valve 19 is closed, the first valve 11 is opened, and the second valve 12 is closed, an confining pressure is applied by the pressure control pump 10 until a set value is reached, and then the first valve 11 is closed, the second valve 12 is opened, and an axial pressure is applied by the pressure control pump 10 until a set value is reached.

Fluid introduction: A fifth valve 25 is opened, a CO₂ gas is fed into a heating device 3 for heating, where a set temperature of the heating device 3 is a formation temperature of a study region, the heated gas enters a purifier 4 for purification, and then enters the first container 5. In this case, whether a pressure numerical value is displayed on a first pressure gauge 7 on a first container 5 (if the pipeline is damaged and has leakage, the pressure value cannot be displayed) is observed. When the pressure value of the first pressure gauge 7 no longer changes, according to the test design, the gas in the first container 5 is pushed into the core holder by the power pump 6, and a temperature detection device 9 (thermoprobe) is inserted into the core to detect whether the temperature is close to the temperature in the heating device 3, thus determining whether the gas has entered a cavity of the core holder. If the temperature on the temperature detection device 9 is close or equal to that of the heating device, it is indicated that the gas has entered the cavity.

Displacement test: The CO₂ gas is used for carrying out the displacement test to displace the displacement fluid (the displacement fluid includes CO₂ and shale oil), and the displacement fluid produced by displacement in the core holder is introduced into the gas-liquid separator 20 for gas-liquid separation, and the fourth valve 21 is opened to observe whether there is a numerical value of the gas flowmeter 22. If there is a numerical value of the gas flowmeter, it is indicated that the displacement test is in normal operation. When the displacement work is carried out for 5 min, 10 min and 15 min, respectively, the nuclear magnetic resonance instrument 1 is turned on to test a T2 spectrum of the core in the core holder (if the pressure needs to be reduced, the first pressure relief valve 16 and the second pressure relief valve 17 are adjusted). When the numerical value of the gas flowmeter 22 no longer changes, it is indicated that the displacement test is over. In this case, the power pump 6 is stopped, and the first valve 11, the second valve 12, the fourth valve 21 and the fifth valve 25 are closed. The numerical value of the gas flowmeter 22 is read, the gas-liquid separator 20 is dismounted, and the liquid in the gas-liquid separator 20 is metered.

Specific examples are used herein for illustration of the principles and embodiments of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. A displacement test device, comprising:
a displacement fluid system having a fluid outlet;
a core holder having a core arranged therein and a fluid inlet in communication with the fluid outlet of the displacement fluid system; and
a nuclear magnetic resonance instrument having a detection cavity, the core holder being arranged in in the detection cavity
wherein the displacement fluid system transports a displacement fluid into the core holder, and the core holder displaces a target fluid from the core.

2. The displacement test device according to claim 1, wherein the displacement fluid system comprises:
a power pump, a material storehouse, a heating device, a purifier, and a first container, all of which are connected and in communication in sequence; wherein an output end of the power pump communicates with the first container, and a first pressure gauge is arranged on the first container.

3. The displacement test device according to claim 2, wherein the core holder comprises:
a holder body, the core being arranged in the holder body such that the holder body is capable of applying an axial pressure and a confining pressure to the core
a temperature detection device having a detection end located in the holder body, the temperature detection device detecting a temperature in the holder body, and
a pressure control assembly connected to the holder body, and controls a magnitude of the confining pressure and the axial pressure of the holder body exerted on the core.

4. The displacement test device according to claim 3, wherein the pressure control assembly comprises:
a pressure control pump having an output end,
a first valve,
a second valve,
a main pipeline having one end in communication with the output end of the pressure control pump,
a first branch pipe having one end in communication with the main pipeline, and another end in communication with a confining pressure regulation port of the holder body, the first valve being arranged on the first branch pipe, and
a second branch pipe having one end, away from the pressure control pump, in communication with the main pipeline, and another end in communication with an axial pressure regulation port of the holder body, the second valve being arranged on the second branch pipe.

5. The displacement test device according to claim 4, wherein the pressure control pump is a manual pump.

6. The displacement test device according to claim 4, wherein the pressure control assembly further comprises:
a first pressure relief pipeline having one end in communication with the first branch pipe between the first value and the holder body, and another end having a first pressure relief valve, and
a second pressure relief pipeline having one end in communication with the second branch pipe between the second valve and the holder body, and another end having a second pressure relief valve.

7. The displacement test device according to claim 4, wherein the pressure control assembly further comprises:
a second container,
a third branch pipe having one end in communication with the main pipeline and another end in communication with the second container, and
a third valve arranged on the third branch pipe.

8. The displacement test device according to claim 1, further comprising a fluid treatment system, wherein the fluid treatment system comprises:
a gas-liquid separator in communication with the fluid output end of the core holder and having a gas output end,
a fourth valve in communication with the gas output end of the gas-liquid separator, and
a gas flowmeter in communication with the fourth valve.

9. The displacement test device according to claim 8, wherein the fluid treatment system further comprises a back pressure valve, the fluid output end of the core holder communicates with the back pressure valve, and the back pressure valve communicates with a fluid inlet of the gas-liquid separator.

10. A displacement test method, comprising:
initial detection including:
placing a treated core in a core holder, and
detecting pore size distribution of a core without displacement test by a nuclear magnetic resonance instrument; and
a test stage including:
displacing a fluid into the core holder through a displacement fluid system,
displacing a target fluid from the core by using the displacement fluid, and
detecting the pore size distribution of the core by using the nuclear magnetic resonance instrument.
